(19) United States
(12) Patent Application Publication
(10) Pub. No.: US 2010/0294987 A1
(43) Pub. Date: Nov. 25, 2010

(54) STABLE, TRANSPORTABLE DECONTAMINATION SYSTEM

(76) Inventor: Jessie Kater, San Diego, CA (US)

Correspondence Address: SQUIRE SANDERS & DEMPSEY LLP, 1 East Washington Street, SUITE 2700, PHOENIX, AZ, 85004, US

(21) Appl. No.: 12/630,527

(22) Filed: Dec. 3, 2009

Related U.S. Application Data
(63) Continuation-in-part of application No. 12/471,231, filed on May 22, 2009.
(60) Provisional application No. 61/262,863, filed on Nov. 19, 2009.

(51) Int. Cl. C01B 15/00; C07C 409/26

(52) U.S. Cl. 252/186.42; 562/2

(57) ABSTRACT

The invention relates to a dry composition that can be added to a liquid (preferably water and/or brackish water) and used to decontaminate a toxin present in a polar or non-polar matrix.

### RELATED APPLICATIONS

This application claims priority to U.S. application Ser. No. 12/471,231 entitled “Broad Spectrum Decontaminate and Dispersement System” filed on May 22, 2009 and to U.S. Provisional Application Ser. No. 61/262,863 filed on Nov. 19, 2009.

### FIELD OF THE INVENTION

The present invention relates to a substance for decontaminating toxins present in the air or on a surface.

### BACKGROUND AND SUMMARY OF THE INVENTION

It is known that reactive species of oxygen (hereafter, “oxygen radicals” or “ROₓ”) render chemical and/or biological agents (collectively, “toxins” and singularly, “toxin”) inactive, which is referred to herein as “decontamination.” A substance that decontaminates toxins is referred to herein as a “decontaminant.”

The present invention is a decontaminant designed to decontaminate one or more of a chemical agent (such as a chemical warfare agent) and a biological agent (such as a biopathogen). The decontaminant utilizes a chemical activation agent, rather than energy from a plasma arc, ultraviolet light or other such energy source, to generate excess oxygen radicals. It is designed to work in an open environment (such as outdoors, rather than in an environmentally-controlled space) and at temperatures ranging from about −25° F. to 125° F. A decontaminant according to the invention can be used in many applications, including on irregular surfaces and/or to destroy toxins in the air.

In one preferred embodiment, the decontaminant includes: (i) a mixture of organic and aqueous liquids that solubilize toxins, and (ii) one or more activators that generate additional reactive oxygen species when reacted with an oxidizing agent, which hydrolyze (and thus decontaminate) toxins, and one or more oxidizing agents. This decontaminant may be deployed utilizing any suitable method or device for one or both of surface decontamination and aerosol decontamination.

A decontaminant according to the invention can be formulated to meet the Joint Program Executive Office for Chemical and Biological Defense (“JPEO”) requirements and be effective against chemical agents including nerve and blister agents (V class, G class and H class) as well as bacteria, viruses and other toxins. Additionally, it can be formulated to be effective over a temperature range of −25° F. to 125° F.

Constituents that may be used in the practice of the invention are described below:

### Solvent

A decontaminant according to the invention may include an organic solvent to solubilize chemical agents (which typically have a non-polar matrix) and/or an aqueous solvent (preferably water) to solubilize biological agents (which typically are soluble in a polar, such as an aqueous, matrix). The toxins are solubilized by the solvent in order to increase the efficiency of the decontamination. The organic solvent is preferably one or more of ethanol, 2,3 butane diol, and 1-hexanol, although any suitable organic solvent(s) may be used.

### Oxygen Radical Donators

A decontaminant according to the invention preferably includes one or more oxygen radical donators. An oxygen radical donator (ROₓ) that may be used with the invention can be hydrogen peroxide as a component of the starting mixture or hydrogen peroxide or other oxygen moieties generated via a chemical reaction, as understood by those skilled in the art. Other suitable oxygen radical donators may be used.

### Activators Providing Additional Oxygen Radicals

One or more activators are included in a decontaminant according to the invention. The activators(s) generate additional reactive oxygen species when reacted with the one or more oxygen radical donators (such as hydrogen peroxide). In a preferred embodiment, hydrogen peroxide and the activator react to create peroxyacetic acid (PAA), which is an oxidizing agent. A preferred activator is tetra acetyl ethylene diamine (TAED), although any suitable activator(s) may be used. The activator(s) (sometimes referred to herein as a “dry activator”) is preferably dry and in a powder form.

### Buffer

One or more buffers may be used to increase the pH of the composition forming the decontaminant and the buffer is preferably NaOH provided in dry, powder form or in a 5.5 Molar (M) solution. The pH is preferably raised to at least about 6.8, most preferably to about 7.1-7.2, and preferably no higher than about 7.5, in order to better facilitate the reaction between the oxygen radical donator(s) and the activator(s). If used, the buffer may be any suitable material(s) for raising the pH to a desired level.

### Oxidizing Agents

One or more oxidizing agents (sometimes referred to herein as a “liquid activator”) are included in a decontaminant according to the invention. The oxidizing agent includes reactive oxygen species and is used to decontaminate the contaminant. An oxidizing agent according to the invention may include PAA, although any suitable oxidizing agent(s) may be used.

### System

The various constituents described above are shipped and stored in separate containers and mixed to form a decontaminant prior to use. In one embodiment, the constituents are shipped and stored in four containers, with one container including the solvent (preferably water and an organic solvent) and the oxygen radical donator (preferably hydrogen peroxide), one including the oxidizing agent (preferably PAA), one including a liquid activator (preferably 5.5 M NaOH) and one including the dry activator (preferably TAED).

A decontaminant according to the invention may be dispersed into the air or onto a surface and any suitable method or device may be utilized for this purpose. Any nozzle that generates relatively uniform particles that can remain suspended in the air may be used.

A preferred decontaminant according to the invention is set forth on the following page:

TABLE I
Components for a Preferred Embodiment of a Decontaminant
PART 1 - Liquid

| Compound | mL | % by Volume |
|---|---|---|
| Ethanol or 2,3 butane diol | 500 mL | 52.66 |
| 1-Hexanol | 50 mL | 5.19 |
| Hydrogen Peroxide and/or | 77.5 mL | 7.99 |
| organic peroxides | | |
| Water | 321.5 mL | 34.16 |
| Totals | 949 mL | 100% |

PART 2 - Dry Activator

| Compound | g | % by Volume |
|---|---|---|
| TAED | 45.0 g | — |
| NaOH (dry powder) | 20.0 g ** | — |

PART 3 - Liquid Activator

| Compound | mL | % by Volume |
|---|---|---|
| Peroxyacetic Acid | 19.97 mL | 39.00% |
| Acetic Acid | 31.23 mL | 61.00% |
| Totals | 51.2 mL | 100% |

After this 3-part chemistry system shown in Table I is mixed and ready to use, the decontaminant formula would be similar to that described below in Table II.

TABLE II
Approximate Composition of the Decontaminant after
mixing the constituents listed in Table I.

| Deployable BIT ™-500 Final Mix | % of Final Volume when mixed |
|---|---|
| Ethanol or 2.3 butaine doil | 43.92 |
| Water | 34.91 |
| 1-Hexanol | 4.29 |
| Hyrdorgen Peroxide | 6.87 |
| Peroxyacetic acid | 1.71 |
| Acetic Acid | 2.68 |
| DAED (by-product) | 3.9 |
| NaOH* (dissolved) | 1.72 |

*NaOH powder is preferably mixed in water to a total amount of 100 mL before placement into the decontaminant. The alternative is to transport 100 mL of 5.5 Molar NaOH.

Below is a description of a preferred embodiment of a dry formulation that can be mixed with liquid (preferably water and/or brackish water) to create a decontaminate according to another aspect of the invention.

A dry formulation according to the invention includes dry constituents to generate peroxyacetic acid when mixed with water. It is preferred that the dry formulation includes carbamide peroxide and TAED, which will generate peroxyacetic acid when mixed with water. The dry formulation also preferably includes a constituent that will raise the pH of the dry mixture/water decontaminate, preferably to about 7, because the conversion of chemicals to peroxyacetic acid is optimal at about that pH level, and as the peroxyacetic acid is being formed it lowers the pH of the decontaminate. A preferred dry constituent for raising the pH level is NaOH, although any suitable material(s) can be used.

The dry formulation also preferably includes a surfactant that will at least partially solubilize a non-polar matrix that may be present in chemical or biochemical contaminants. In that manner, the decontaminate containing the peroxyacetic acid will better neutralize chemical or biochemical contaminants present within a non-polar matrix. A preferred surfactant is sodium dodecyl sulfate.

Finally, it is preferred that the dry mixture also include an antifoaming agent to prevent the surfactant from generating too much, or any, foam or suds. A preferred anti-foaming agent is Antifoam-A, made by Sigma Aldrich, located in St Louis Mo.

Any suitable water source can be used, including distilled water, fresh water, or brackish water (which includes salt water). If water containing salt is used it may be treated with a chealating agent first, such as Chelex-100, manufactured by Bio-Rad Laboratories, Life Science Group located at 2000 Alfred Nobel Drive, Hercules, Calif. 94547. If used, the amount of chealating agent per volume of water is determined by the salinity of the water.

When the dry formulation is added to water, the carbamide peroxide and TAED react to form peroxyacetic acid, which will decontaminate a chemical or biological contaminant and the surfactant (shown here as preferably sodium dodecyl sulfate) breaks apart oily-based contaminants (i.e., those in a non-polar matrix) to allow them to be decontaminated. As used herein, “decontaminates” or “decontaminated” means to be decontaminated to at least some degree.

Following is one preferred mixture of dry formulation and water:

| Dry Mixture Components | Weight (g) |
|---|---|
| Sodium dodecyl sulfate | 30.01 |
| Antifoam-A | 2.40 |
| carbamide peroxide | 207.08 |
| Tetraacetylethylenediamine | 120.05 |
| (TAED) | |
| Sodium Hydroxide (NaOH) | 10.20 |
| Total weight | 369.75 |
| Water | 630.252 |
| Total weight water + dry mixture | 1000 |

Below are preferred ranges of dry constituents that may be used to form a decontaminant according to the invention:

| | Most Preferred | Effective | |
|---|---|---|---|
| | Weight (g) | range (g) | |
| Dry Mixture Components | Weight (g) | Low (g) | High (g) |
| Sodium dodecyl sulfate | 30.01 | 3 | 90 |
| Antifoam-A | 2.40 | 0.15 | 30 |
| carbamide peroxide | 207.08 | 60 | 240 |
| Tetraacetylethylenediamine | 120.05 | 60 | 170 |
| (TAED) | | | |
| Sodium Hydroxide (NaOH) | 10.20 | 7.2 | 11.2 |
| Total weight | 369.75 | 130.35 | 541.2 |
| Water | 630.252 | 869.65 | 458.8 |

| | |
|---|---|
| Total weight water + dry mixture | 1000 |

Below are preferred mixing instructions for making a decontaminant using a preferred mixture:
Add about 370 grams of dry formulation to a vessel, such as a beaker or pressure release container.
Add about 630 grams of water from any suitable local water source.
Agitate until the decontaminant solution is clear, which means that no dry formulation particles are visible to the eye in the decontaminate solution (the agitation may take up to approximately 20 minutes).
The water and dry formulation may be mixed, and the resulting decontaminant used, at any workable temperature, with a preferred range being between 32 to 150 degrees F.
Anti-freezing agents, such as MgCl₂, may be added to the decontaminant to lower the operating temperature to about 10 degrees F.
A decontaminant according to the invention preferably has no non-polar solvent.

Having thus described some embodiments of the invention, other variations and embodiments that do not depart from the spirit of the invention will become apparent to those skilled in the art. The scope of the present invention is thus not limited to any particular embodiment, but is instead set forth in the appended claims and the legal equivalents thereof. Unless expressly stated in the written description or claims, the steps of any method recited in the claims may be performed in any order capable of yielding the desired result.

What is claimed is:

1. A formulation of dry components that can be added to a liquid to form peroxyacetic acid.

2. The formulation of claim 1 that comprises carbamide peroxide and TAED.

3. The formulation of claim 1 that further includes a surfactant.

4. The formulation of claim 2 that further includes a surfactant

5. The formulation of claim 3 wherein the surfactant comprises sodium dodecyl sulfate.

6. The formulation of claim 4 wherein the surfactant comprises sodium dodecyl sulfate

7. The formulation of claim 5 that further includes an antifoaming agent.

8. The formulation of claim 6 that further includes an antifoaming agent

9. The formulation of claim 7 wherein the antifoaming agent comprises Antifoam-A.

10. The formulation of claim 8 wherein the antifoaming agent comprises Antifoam-A

11. The formulation of claim 1 that further includes a constituent to increase the pH level.

12. The formulation of claim 2 that further includes a constituent to increase the pH level

13. The formulation of claim 11 wherein the material to increase the pH comprises NaOH.

14. The formulation of claim 12 wherein the material to increase the pH comprises NaOH.

15. The formulation of claim 1 that does not include peroxyacetic acid.

16. The formulation of claim 2 that has between 37.1%-46% TAED by weight.

17. The formulation of claim 2 that has between 44.3%-46% carbamide peroxide by weight.

18. The formulation of claim 3 that has between 2.3%-19.6% surfactant by weight.

19. The formulation of claim 18 wherein the surfactant is sodium dodecyl sulfate.

20. The formulation of claim 7 that includes 0.12%-5.5% antifoaming agent.

21. The formulation of claim 20 wherein the antifoaming agent is Antifoam-A.

22. The formulation of claim 11 that includes between 2.1% and 5%% of a constituent to raise the pH when the formulation is added to water.

23. The formulation of claim 22 wherein the constituent comprises NaOH.

24. A formulation of dry constituents for mixing with liquid to form a decontaminant, the formulation comprising by weight about: 2.3%-19.6% surfactant, 0.12%-5.5% antifoaming agent, 44.3%-46% carbamide peroxide, 37.1%-46% TAED and 2.1%-5.5% of a constituent for raising the pH when the formulation is added to water.

25. The formulation of claim 24 wherein the surfactant comprises sodium dodecyl sulfate.

26. The formulation of claim 24 wherein the antifoaming agent comprises Antifoam-A.

27. The formulation of claim 24 wherein the constituent for raising the pH comprises NaOH.

28. A decontaminate including water and/or brackish water and the formulation of claim 1.

29. The decontaminant of claim 28 that comprises carbamide peroxide and TAED.

30. The decontaminant of claim 28 that further includes a surfactant.

31. The decontaminant of claim 29 that further includes a surfactant

32. The decontaminant of claim 30 wherein the surfactant comprises sodium dodecyl sulfate.

33. The decontaminant of claim 31 wherein the surfactant comprises sodium dodecyl sulfate

34. The decontaminant of claim 30 that further includes an antifoaming agent.

35. The decontaminant of claim 31 that further includes an antifoaming agent

36. The decontaminant of claim 34 wherein the antifoaming agent comprises Antifoam-A.

37. The decontaminant of claim 35 wherein the antifoaming agent comprises Antifoam-A

38. The decontaminant of claim 28 that further includes a constituent to increase the pH level.

39. The decontaminant of claim 29 that further includes a constituent to increase the pH level

40. The decontaminant of claim 38 wherein the material to increase the pH comprises NaOH.

41. The decontaminant of claim 39 wherein the material to increase the pH comprises NaOH.

42. The decontaminant of claim 28 that does not include peroxyacetic acid before the water and/or brackish water is mixed with the formulation.

43. The decontaminant of claim 28 that includes by weight about: 2.3%-19.6% surfactant, 0.12%-5.5% antifoaming agent, 44.3%-46% carbamide peroxide, 37.1%-46% TAED, 2.1%-5.5% of a constituent to raise the pH, and about 63% water and/or brackish water.

44. The decontaminant of claim 43 wherein the surfactant comprises sodium dodecyl sulfate.

45. The decontaminant of claim 43 wherein the constituent to raise the pH comprises NaOH.

46. The decontaminant of claim 43 wherein the antifoaming agent is Antifoam-A.

47. The decontaminant of claim 43 that includes about: 3-90 grams of surfactant, 0.15-30 grams of antifoaming agent, 6-240 grams of carbamide peroxide, 60-170 grams of TAED, and 7.2 to 11.2 grams of NaOH to every about 630 grams of water and/or brackish water.

48. The decontaminant of claim 28 that is formed by agitating the formulation in the water and/or brackish water for about 20 minutes.

49. The decontaminant of claim 28 that does not include a non-polar solvent.

50. The decontaminant of claim 28 that includes between about 130.35 to 541.2 grams of formulation to every about 458.8 to 869.65 grams of water and/or brackish water.

51. The decontaminant of claim 28 that includes a chealating agent.

52. The decontaminant of claim 51 wherein the chealating agent is Chelex-100.

53. The decontaminant of claim 28 that has a pH of about 7.

54. The decontaminant of claim 52 wherein the chealating agent is added to the water before the formulation.

55. The decontaminant of claim 28 that further includes an anti-freezing agent.

56. The decontaminant of claim 55 wherein the anti-freezing agent is MgCl₂.
